(19) United States
(12) Patent Application Publication
(10) Pub. No.: US 2015/0252346 A1
(43) Pub. Date: Sep. 10, 2015

(54) METHOD FOR ISOLATING UROKINASE ENZYME FROM AMNIOTIC FLUID

(71) Applicant: Khushal C. Bhaiji, Hinckley, OH (US)

(72) Inventor: Khushal C. Bhaiji, Hinckley, OH (US)

(21) Appl. No.: 14/198,640

(22) Filed: Mar. 6, 2014

(51) Int. Cl. C12N 9/72

(52) U.S. Cl. CPC C12N 9/6462

(57) ABSTRACT

A method for isolating urokinase enzyme from amniotic fluid. The method includes the steps of: contacting the amniotic fluid containing the urokinase enzyme and contaminates with a library of binding moieties having different binding moieties; binding the amniotic fluid and the contaminates to the library of binding moieties leaving the urokinase enzyme unbound so as to form bound amniotic fluid, bound contaminates, and unbound urokinase enzyme; separating the unbound urokinase enzyme from the bound amniotic fluid and the bound contaminates; and collecting the unbound urokinase enzyme while leaving the bound contaminates and the bound amniotic fluid in a flow-through.

### BACKGROUND OF THE INVENTION

A. Field of the Invention

The embodiments of the present invention relate to a method for isolating urokinase enzyme, and more particularly, the embodiments of the present invention relate to a method for isolating urokinase enzyme from amniotic fluid.

B. Description of the Prior Art

(1) Urokinase Enzyme and Amniotic Fluid.

Urokinase enzyme is well known as a thrombolytic (clot buster) for intravenal use, most notably in the treatment of stenosis of coronary arterie(s) during a cardiac infarction (heart attack).

A usual commercial source from which the urokinase enzyme has been isolated and purified for commercial clinical use is neonatal (newly born) kidneys, as reported by Abbott Laboratories and by others. The use of neonatal kidneys as the source encountered humanitarian objections and was banned from pharmaceutical use by the United States Food and Drug Administration (“FDA”).

Kidneys of neonatal cats became a next source for the urokinase enzyme. Animal lovers soon thereafter raised loud protests alleging cruelty to the cats and to cat-farming, whereupon cat neonatal kidneys have been rejected in the United States of America as an acceptable source of the urokinase enzyme.

In Canada, the urokinase enzyme is still marketed by Microbix Biosystems, Inc. (“Microbix”) under the brand name KIN-LITIC. Microbix is located at 265 Watline Avenue, Mississaugaj, Ontario Canada, telephone number (416) 234-1624, and fax number (416) 234-1626. Techniques, processes, and equipment for gathering, isolating, purifying, storing, reactivating, and using the urokinase enzyme are commercially available in state-of-the-art manufacturing and clinical processes and procedures. Microbix currently performs the manufacturing, is quite proficient in extracting and refining the urokinase enzyme commercially, and is a commercial supplier of urokinase enzyme powder to the pharmaceutical industry and clinics. No significant, unusual, complex, or unmanageable hazards have been, or are currently, associated with Microbix's production of the urokinase enzyme powder.

A principle active ingredient of the urokinase enzyme is a low molecular weight form of the urokinase enzyme, which consists of an A chain of about 2,000 daltons linked by a sulfhydyl bond to a B chain of about 30,400 daltons. Pharmaceutical-grade urokinase enzyme is supplied commercially as a sterile, lyophilized, and white powder containing 250,000 IU of the urokinase enzyme per vile, mannitol (25 mg/vile), human albumin (250 mg/vial), and sodium chloride (50 mg/vile). The product is stored at 2° C. to 8° C. Following reconstitution, the urokinase enzyme product is a clear and slightly straw-colored solution. Each ml. contains 50,000 IU of the urokinase enzyme activity.

The urokinase enzyme usually contains no anti-microbial preservatives, preferably, it is reconstituted just prior to its use. The urokinase enzyme is reconstituted by adding 5 ml. of water for injection or 0.2 micron filtered and purified water per vial. After reconstitution, each vial of the urokinase enzyme is inspected for discoloration and presence of particulate material. The solution should be pale and straw-colored. Highly colored solution should be rejected.

Thin and translucent filaments may occasionally occur in vials of reconstituted urokinase enzyme, but these filaments do not indicate any decrease in potency of the urokinase enzyme product or any other defect. To minimize formation of the filaments, shaking the vial during reconstitution is avoided. Rather, the vial is rolled and tilted to enhance reconstitution. The solution may be terminally filtered, for example, through a 0.45 micron or smaller cellulose membrane filter.

The urokinase enzyme is clinically of great importance in saving lives. Yet sourcing the urokinase enzyme from neonatal kidneys was a major obstacle in its acceptance. Society is aware that unethical people could—and likely did—farm and kill babies and then kittens for their kidneys. Neonatal kidneys became unacceptable as a source for urokinase enzyme, and it was banned by the FDA. Producers of the urokinase enzyme who adopted kidneys of cats as the source were hounded by animal lovers.

Inside a pregnant woman's uterus is an amniotic sack that contains amniotic fluid and a growing fetus. The amniotic fluid is important for several reasons:
Amniotic fluid helps keep the fetus warm;
Amniotic fluid provides lubrication that keeps the body parts of the fetus from growing together because the fetus' parts are growing quickly, but in some case fingers and toes can become webbed as a result of not enough amniotic fluid circulating in the uterus;
Amniotic fluid helps the fetus' lungs to develop;
Amniotic fluid enables the fetus to move easily so that he or she can exercise his or her muscles and strengthen his or her bones before he or she is born; and
Amniotic fluid acts like a shock absorber for the fetus by distributing any force that might push on the mother's uterus.

Amniotic fluid is 98% water and 2% salts and cells from the fetus, Until the fetal kidneys start working during month four of pregnancy, the amniotic fluid is made by the mother's body. After month four, the fetus starts to make his or her contribution to the amniotic fluid by urinating into it. The urine in the amniotic sack is harmless to the fetus. The fetus swallows the amniotic fluid, which then passes through his or her digestive system, into his or her kidneys, and back out again to the amniotic sack as urine. In this way, the fetus practices using his or her digestive and urinary systems before he or she is born. And, in this way the fetus enriches the amniotic fluid's urine content. Doctors can tell by the amount of amniotic fluid whether the fetus is having difficulty with his or her swallowing reflex.

Amniotic fluid is a product of a fascinating cooperation between a mother and her fetus. Amniotic fluid as a source for the urokinase enzyme adds to its unique role in the life cycle of humans. Thus, there exists a need for a method for isolating urokinase enzyme from amniotic fluid.

(2) Derived Cell Products.

Numerous innovations for derived cell products have been provided in the prior art, which will be described below in chronological order to show advancement in the art, and which are incorporated herein in their entirety by reference thereto. Even though these innovations may be suitable for the specific individual purposes to which they address, nevertheless, they differ from the embodiments of the present invention in that they do not teach a method for isolating urokinase enzyme from amniotic fluid.

(a) U.S. Pat. No. 5,653,996 to Hsu.

U.S. Pat. No. 5,653,996—issued to Hsu on Aug. 5, 1997 in U.S. class 424 and subclass 450—teaches methods for preparing liposomes utilizing aerosolization of a solution including bilayer-forming materials and optional additional molecules onto an aqueous surface. The aerosolization is mist spraying through a frequency-generated vibrating nozzle.

(b) United States Patent Application Publication Number US 2003/0235563 A1 to Strom et al.

United States Patent Application Publication Number US 2003/0235563 A1—published to Strom et al. on Dec. 25, 2003 in U.S. class 424 and subclass 93.21—teaches placental-derived stem cells and methods and compositions for therapeutic uses of placental-derived stem cells or placental-derived stem cells that have been induced to differentiate into a desired tissue type into a recipient host in amounts sufficient to result in production of the desired cell type, e.g., hepatic, pancreatic, neuronal, or nervous tissue.

(c) United States Patent Application Publication Number US 2004/0161419 A1 to Strom et al.

United States Patent Application Publication Number US 2004/0161419 A1—published to Strom et al. in U.S. class 424 and subclass 93.21—teaches placental stem cells and methods and compositions for therapeutic uses of placental stem cells or placental stem cells mat have been induced to differentiate into a desired tissue type into a recipient host in amounts sufficient to result in production of the desired cell type, e.g., hepatocytes, neural cells, pancreatic cells, vascular endothelial cells, or cardiomyocytes.

(d) United States Patent Application Publication Number US 2007/0122903 A1 to Rezania et al.

United States Patent Application Publication Number US 2007/0122903 A1—published to Rezania et al. on May 31, 2007 in U.S. class 435 and subclass 325—teaches an expandable population of amniotic fluid-derived cells that are differentiate into a β-cell lineage and methods for isolating and expanding these amniotic fluid-derived cells, as well as related methods and compositions for utilizing these cells in the therapeutic treatment of diabetes.

(e) U.S. Pat. No. 7,754,861 B2 to Boschetti et al.

U.S. Pat. No. 7,754,861 B2—issued to Boschetti et al. on Jul. 13, 2010 in U.S. class 530 and subclass 412—teaches methods and kits for purifying a target protein group. The method includes the steps of contacting a sample including at least 95% of the target protein group and at most 5% of contaminating proteins with a library of binding moieties having different binding moieties, binding the contaminating proteins and a minority of the target protein group to the library of binding moieties, separating the unbound target protein group from the proteins bound to the library of binding moieties, and collecting the unbound target protein. The collected target protein is more pure than the target protein group in the sample.

It is apparent that numerous innovations for providing derived cell products have been provided in the prior art, which are adapted to be used. Furthermore, even though these innovations may be suitable for the specific individual purposes to which they address, nevertheless, they would not be suitable for the purposes of the embodiments of the present invention; as heretofore described, namely, a method for isolating urokinase enzyme from amniotic fluid.

### SUMMARY OF THE INVENTION

Thus, an object of the embodiments of the present invention is to provide a method for isolating urokinase enzyme from amniotic fluid, which avoids the disadvantages of the prior art.

Briefly stated, another object of the embodiments of the present invention is to provide a method for isolating urokinase enzyme from amniotic fluid, which includes the steps of: contacting the amniotic fluid containing the urokinase enzyme and contaminates with a library of binding moieties having different binding moieties; binding the amniotic fluid and the contaminates to the library of binding moieties leaving the urokinase enzyme unbound so as to form bound amniotic fluid, bound contaminates, and unbound urokinase enzyme; separating the unbound urokinase enzyme from the bound amniotic fluid and the bound contaminates; and collecting the unbound urokinase enzyme while leaving the bound contaminates and the bound amniotic fluid in a flow-through.

The novel features considered characteristic of the embodiments of the present invention are set forth in the appended claims. The embodiments of the present invention themselves, however, both as to their construction and to their method of operation together with additional objects and advantages thereof will be best understood from the following description of the embodiments of the present invention when read and understood in connection with the accompanying figures of the drawing.

### BRIEF DESCRIPTION OF THE FIGURES OF THE DRAWING

The figures of the drawing are briefly described as follows:

FIG. 1 is an article entitled MOM'S CLOT RISKS LONGER; Health & Science section of Newsday; Feb. 14, 2014; page 45; and

FIGS. 2A-2B are a flow chart of the method for isolating urokinase enzyme from amniotic fluid.

### LIST OF REFERENCE NUMERALS UTILIZED IN THE FIGURES OF THE DRAWING

10 method for isolating urokinase enzyme 12 from amniotic fluid 14
12 urokinase enzyme
14 amniotic fluid
16 contaminates,
18 library of binding moieties
20 bound amniotic fluid
22 bound contaminates
24 unbound urokinase enzyme

### DETAILED DESCRIPTION OF THE PREFERRED EMBODIMENTS

A. Amniotic Fluid as a Source of Urokinase Enzyme.

Applicant Bhaiji teaches to isolate the urokinase enzyme from amniotic fluid. Applicant Bhaiji has discovered and recognized amniotic fluid to be a rich source of the urokinase enzyme.

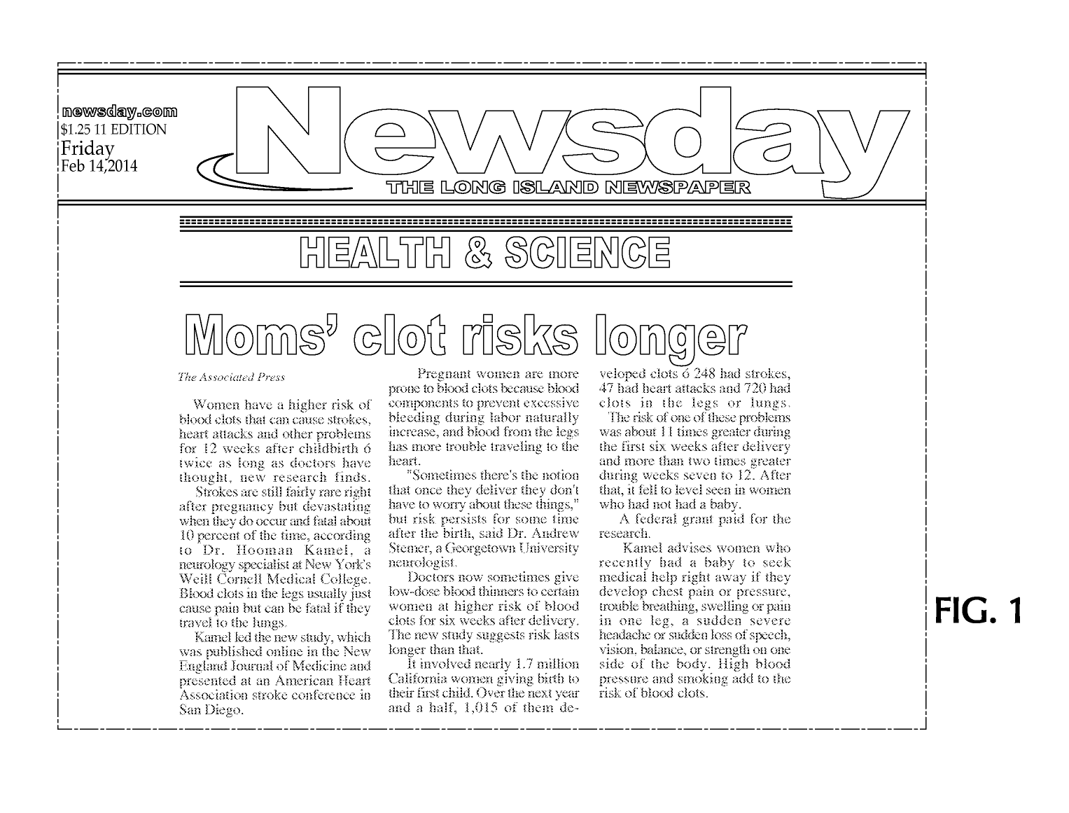

As a clinical physician, Applicant Bhaiji has observed that during childbirth, if even a small amount of the amniotic fluid gets into a mother's bloodstream she bled quite profusely. This emergency situation is known as amniotic fluid embolism. The emergency situation is very dangerous for the mother unless she is saved promptly by a transfusion of blood into each of her arms. Applicant Bhaiji theorized that this emergency situation, from one of several possibilities, is caused by a substantial amount of the urokinase enzyme in the amniotic fluid. Childbirth involves delivery of a child into the world and it also respects the welfare of the child's mother. The amniotic fluid is a potentially serious danger to the mother's delivery of the baby. After delivery and thereby exhaustion of the amniotic fluid and the urokinase enzyme contained therein, the mother's blood clot rate increases. MOM'S CLOT RISKS LONGER; Health & Science; Newsday; Feb. 14, 2014; page 45 (FIG. 1). Yet it has never been discovered until now, that amniotic fluid presents a rich, inexpensive, abundant, and excellent source from which the urokinase enzyme can be made available commercially.

Applicant Bhaiji experienced clinically that amniotic fluid embolism can kill a mother in childbirth. Applicant Bhaiji's clinical experience signaled to him a suspicion that the amniotic fluid may be rich in the urokinase enzyme. The urokinase enzyme can be made available plentifully and provides a non-objectionable, rich, inexpensive, and excellent commercial source for the manufacture of pharmaceutical-grade urokinase enzyme. In view of Applicant Bhaiji's discovery of deriving the urokinase enzyme from amniotic fluid, the urokinase enzyme would now be marketable, more economically, and without any humanitarian or animal-cruelty stigma, whereby the urokinase enzyme can now resume its place as a thrombolytic (clot buster) for coronary artery stenosis during heart attacks and for similar atrial uses.

Applicant Bhaiji's discovery of amniotic fluid (a waste substance) as a rich and inexpensive source of the urokinase enzyme takes away fear of baby-farming and cat-farming and subsequent slaughtering, with related social stigmas or legal prohibition.

Applicant Bhaiji's discovery is not merely substituting one known source for another known source. Applicant Bhaiji is a highly inquisitive and an intensely focused observer of clinical procedures and the pharmaceutical industry. Applicant Bhaiji is far more perceptive, experienced, focused, brilliant, imaginative, creative, and practical than an ordinary artisan clinician and/or researcher.

Applicant Bhaiji has discovered the amniotic fluid answer in a dangerous waste substance that happens to be tragically incidental to childbirth. Applicant Bhaiji found that the amniotic fluid contains about double the concentration of the urokinase enzyme found in a normal adult's kidneys,

B. Method for Isolating Urokinase Enzyme from Amniotic Fluid.

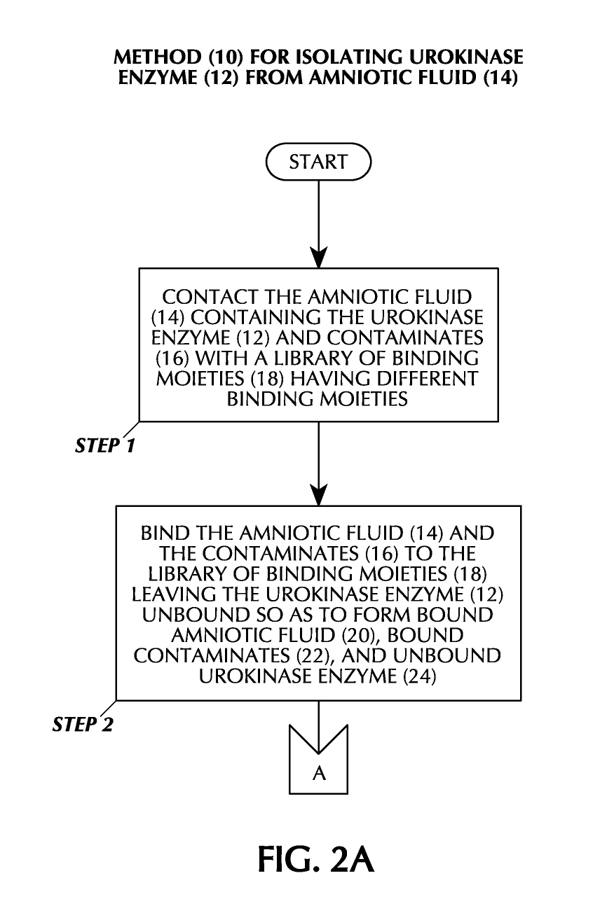

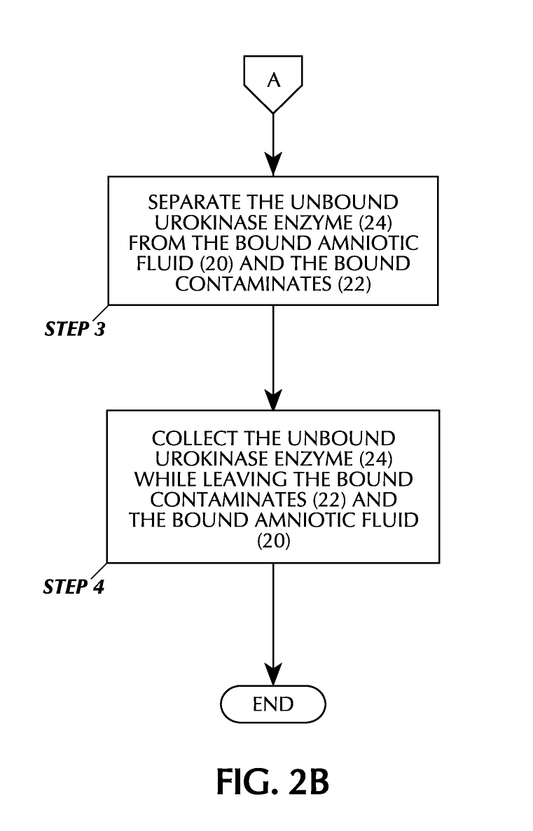

Referring now to FIGS. 2A-2B, which are a flow chart of the method for isolating urokinase enzyme from amniotic fluid, and in which like numerals indicate like parts, the method of the embodiments of the present invention is shown generally at 10 for isolating urokinase enzyme 12 from amniotic fluid 14.

The method 10 for isolating the urokinase enzyme 12 from the amniotic fluid 14 comprises the steps of:
STEP 1: As shown in FIG. 2-A, contacting the amniotic fluid 14 containing the urokinase enzyme 12 and contaminates 16 with a library of binding moieties 18 having different binding moieties;
STEP 2: As shown in FIG. 2-A, binding the amniotic fluid 14 and the contaminates 16 to the library of binding moieties 18 leaving the urokinase enzyme 12 unbound so as to form bound amniotic fluid 20, bound contaminates 22, and unbound urokinase enzyme 24;
STEP 3: As shown in FIG. 2-B, separating the unbound urokinase enzyme 24 from the bound amniotic fluid 20 and the bound contaminate 22; and
STEP 4: As shown in FIG. 2-B, collecting the unbound urokinase enzyme 24 while leaving the bound contaminates 22 and the bound amniotic fluid 20 in a flow-through.

C. The Contaminates 16.

Because the method 10 of the embodiments of the present invention provides the library of binding moieties 18, one does not need any information of the identity or source of the contaminates 16.

D. The Library of Binding Moieties 20.

The library of binding moieties 20 refers to a collection of different binding moieties wherein the binding moiety refers to a chemical moiety that binds an analyte.

The library of binding moieties 20 is either chemically synthesized, harvested from a natural source, or in the case of the library of binding moieties 20 being bio-organic polymers, produced using recombinant techniques.

Each binding, moiety 20 is boundable to at least one solid support, wherein the at least one solid support includes:
Spherical discrete particles;
Irregular discrete particles;
Beads;
Fibers;
Filters;
Membranes, and
Monoliths.

Each binding moiety 20 is attachable to a different solid support, while a plurality of different binding moieties 20 are attachable to a single solid support.
E. The step of contacting the amniotic fluid 14 containing the urokinase enzyme 12 and the contaminates 16 with the library of binding moieties 18 having different binding moieties, and the step of binding the amniotic fluid 14 and the contaminates 16 to the library of binding moieties 18 leaving the urokinase enzyme 12 unbound so as to form bound amniotic fluid 20, bound contaminates 22, and unbound urokinase enzyme 24.

Contacting the amniotic fluid 14 containing the urokinase enzyme 12 and the contaminates 16 with the library of binding moieties 18 having different binding moieties, and binding the amniotic fluid 14 and the contaminates 16 to the library of binding moieties 18 leaving the urokinase enzyme 12 unbound so as to form bound amniotic fluid 20, bound contaminates 22, and unbound urokinase enzyme 24 are accomplished by:
Admixing the amniotic fluid 14 containing the urokinase enzyme 12 and contaminates, 16 with the library of binding moieties 20;
Swabbing the amniotic fluid 14 containing the urokinase enzyme 12 and the contaminates 16 onto the library of binding moieties 20;
Flowing the amniotic fluid 14 containing the urokinase enzyme 12 and the contaminates 16 over a solid support having the library of binding moieties 20 attached thereto;
A suspension batch process;
Passing the amniotic fluid 14 containing the urokinase enzyme 12 and the contaminates, 16 over a column packed with the library of binding moieties 20 attached to a solid support; or
A fluidized bed process.

Contacting the amniotic fluid 14 containing the urokinase enzyme 12 and the contaminates 16 with the library of binding moieties 20 and binding of analytes to the binding moieties 20 is done for a period of time sufficient for binding the amniotic fluid 14 containing the contaminates 16 to the library of binding moieties 20. Typically, the library of binding moieties 20 and the amniotic fluid 14 containing the urokinase enzyme 12 and the contaminates 16 are incubated together for at least about 10 min., usually at least about 20 min., more usually at least about 30 min., and more usually at least about 60 min.

Incubation time may also be for several hours, for example up to 12 hrs, but typically does not exceed about 1 hr. When the method 10 of the embodiments of the present invention is performed, for example, using a column, the time for contacting the amniotic fluid 14 containing the urokinase enzyme 12 and the contaminates 16 with the library of binding moieties 20 is referred to as residence time, which is typically in a range from about 1 minute about 20 minutes.
F. The step of separating the unbound urokinase enzyme 24 from the bound amniotic fluid 20 and the bound contaminate 22, and the step of collecting the unbound urokinase enzyme 24 while leaving the bound contaminates 22 and the bound amniotic fluid 20.

Separating the unbound urokinase enzyme 24 from the bound amniotic fluid 20 and the bound contaminate 22, and the step of collecting the unbound urokinase enzyme 24 while leaving the bound contaminates 22 and the bound amniotic fluid 20 are accomplished by:
Centrifugation;
Column chromatography;
Use of linker moieties; or
Use of magnetic beads.

After binding the bound amniotic fluid 20 and the bound contaminates 22, the result is loaded onto a column that retains the at least one solid support and the bound amniotic fluid 20 and the bound contaminates 22 bound thereto. The unbound urokinase enzyme 24 is present in a flow-through from where the unbound urokinase enzyme 24 is collectable.

G. Clinical Delivery of the Unbound Urokinase Enzyme 24.

The unbound urokinase enzyme 24 is clinically deliverable via:
IV;
Sub lingually;
Intranasally; and
Topically.

H. Impressions.

It will be understood that each of the elements described above, or two or more together, may also find a useful application in other types of constructions differing from the types described above.

While the embodiments of the present invention have been illustrated and described as embodied in a method for isolating urokinase enzyme from amniotic fluid, nevertheless, they are not limited to the details shown, since it will be understood that various omissions, modifications, substitutions, and changes in the forms and details of the embodiments of the present invention illustrated and their operation can be made by those skilled in the art without departing in any way from the spirit of the embodiments of the present invention.

Without further analysis, the foregoing will so fully reveal the gist of the embodiments of the present invention that others can by applying current knowledge readily adapt them far various applications without omitting features that from the standpoint of prior art fairly constitute characteristics of the generic or specific aspects of the embodiments of the present invention.

The invention claimed is:

1. A method for isolating urokinase enzyme from amniotic fluid, comprising the steps of:
a) contacting the amniotic fluid containing the urokinase enzyme and contaminates with a library of binding moieties having different binding moieties;
b) binding the amniotic fluid and the contaminates to the library of binding moieties leaving the urokinase enzyme unbound so as to form bound amniotic fluid, bound contaminates, and unbound urokinase enzyme;
c) separating the unbound urokinase enzyme from the bound amniotic fluid and the bound contaminates; and
d) collecting the unbound urokinase enzyme while leaving the bound contaminates and the bound amniotic fluid in a flow-through.

2. The method of claim 1, wherein the library of binding moieties is chemically synthesized.

3. The method of claim 1, wherein the library of binding moieties is harvested from a natural source.

4. The method of claim 1, wherein the library of binding moieties is produced using recombinant techniques.

5. The method of claim 1, wherein said contacting and said binding steps are accomplished by admixing the amniotic fluid containing the urokinase enzyme and the contaminates with the library of binding moieties.

6. The method of claim 1, wherein said contacting and said binding steps are accomplished by swabbing the amniotic fluid containing the urokinase enzyme and the contaminates onto the library of binding moieties.

7. The method of claim 1, wherein said contacting and said binding steps are accomplished by flowing the amniotic fluid containing the urokinase enzyme and the contaminates over a solid support having the library of binding moieties attached thereto.

8. The method of claim 1, wherein said contacting and said binding steps are accomplished by a suspension batch process.

9. The method of claim 1, wherein said contacting and said binding steps are accomplished by passing the amniotic fluid containing the urokinase enzyme and the contaminates over a column packed with the library of binding moieties attached to a solid support.

10. The method of claim 1, wherein said contacting and said binding steps are accomplished by a fluidized bed process.

11. The method of claim 1, wherein said contacting and said binding steps are done for a period of time sufficient for binding the amniotic fluid containing the contaminates to the library of binding moieties.

12. The method of claim 1, wherein the library of binding moieties and the amniotic fluid containing the urokinase enzyme and the contaminates are incubated together at least about 10 min., usually at least about 20 min., more usually at least about 30 min., and more usually at least about 60 min.

13. The method of claim 1, wherein said contacting step includes contacting the amniotic fluid containing the urokinase enzyme and the contaminates with the library of binding moieties in a range from about 1 minute to about 20 minutes.

14. The method of claim 1, wherein said separating step and said collecting step are accomplished by centrifugation.

15. The method of claim 1, wherein said separating step and said collecting step are accomplished by column chromatography.

16. The method of claim 1, wherein said separating step and said collecting step are accomplished by use of linker moieties.

17. The method of claim 1, wherein said separating step and said collecting step are accomplished by use of magnetic beads.

18. The method of claim 1, wherein each binding moiety is bound to at least one solid support.

19. The method of claim 18, wherein the at least one solid support includes spherical discrete particles.

20. The method of claim 18, wherein the at least one solid support includes irregular discrete particles.

21. The method of claim 18, wherein the at least one solid support includes beads.

22. The method of claim 18, wherein the at least one solid support includes fibers.

23. The method of claim 18, wherein the at least one solid support includes filters.

24. The method of claim 18, wherein the at least one solid support includes membranes.

25. The method of claim 18, wherein the at least one solid support includes monoliths.

26. The method of claim 18, wherein each binding moiety is attached to a different solid support.

27. The method of claim 18, wherein a plurality of different binding moieties are attached to a single solid support.

28. The method of claim 18, wherein the bound amniotic fluid and the bound contaminates are loaded onto a column that retains the at least one solid support and the bound amniotic fluid and the bound contaminates bound thereto, while the unbound urokinase enzyme is present in a flow-through from where the unbound urokinase enzyme is collected.
